(19) United States
(12) Patent Application Publication
(10) Pub. No.: US 2019/0062769 A1
(43) Pub. Date: Feb. 28, 2019

(54) LATEX PRODUCTION TRANSCRIPTION FACTORS AND METHODS OF ENHANCING LATEX PRODUCTION

(71) Applicant: The Regents of the University of California, Oakland, CA (US)

(72) Inventors: Henrik Vibe Scheller, Millbrae, CA (US); Solomon Stonebloom, Alameda, CA (US)

(21) Appl. No.: 16/117,792

(22) Filed: Aug. 30, 2018

Related U.S. Application Data
(60) Provisional application No. 62/553,080, filed on Aug. 31, 2017.

(51) Int. Cl. C12N 15/82; C07K 14/415; C12P 5/02

(52) U.S. Cl. CPC C12N 15/8243; C07K 14/415; C12N 15/8261; C12P 5/026; C12N 15/8257

(57) ABSTRACT

The invention provides methods of engineering latex-producing plants, e.g., guayule plants, to increase latex production. The invention additionally provides plants engineered in accordance with the invention and methods of using the plants to produce latex.

### CROSS-REFERENCE TO RELATED APPLICATIONS

This application claims priority to U.S. provisional application No. 62/553,080, filed Aug. 31, 2017, which is herein incorporated by reference.

### STATEMENT AS TO RIGHTS TO INVENTIONS MADE UNDER FEDERALLY SPONSORED RESEARCH AND DEVELOPMENT

This invention was made with government support under Contract No. DE-AC02-05CH11231 awarded by the U.S. Department of Energy. The government has certain rights in this invention.

### REFERENCE TO A “SEQUENCE LISTING” SUBMITTED AS ASCII TEXT FILES VIA EFS-WEB

The Sequence Listing written in the ASCII text file 077429-015310US-1103305_SequenceListing.txt created on Aug. 29, 2018, 10,055 bytes, machine format IBM-PC, MS-Windows operating system, in accordance with 37 C.F.R. §§ 1.821- to 1.825, is hereby incorporated by reference in its entirety for all purposes.

### BACKGROUND OF THE INVENTION

Natural rubber is made from the latex exudate of the Para rubber tree (Hevea brasiliensis) and is used extensively in many industrial and commercial applications. While synthetic rubber (polyisoprene) is capable of substituting for natural rubber in some applications, the strength and thermal stability of natural, enzymatically polymerized rubber are critical in applications such as aviation and trucking tires. Currently, rubber is produced nearly exclusively in Southeast Asia. Economically viable rubber production from Hevea is dependent on inexpensive labor in developing countries and is threatened by a fungal rust that has prevented all efforts at large-scale production of rubber in the Americas. New sources of natural rubber would be advantageous for improving price and supply stability of this important industrial material. The North American desert shrub Guayule (Parthenium argentatum) was used to produce rubber by the Aztecs in northern Mexico and was cultivated widely during WWII when the supply of rubber from Malaysia was cut-off by the Japanese Pacific Blockade (Hammond & Polhamus, Research on guayule (Parthenium argentatum), 1942-1959, 1965). However, rubber production in guayule remains uncompetitive with production in Hevea.

In Hevea, latex is produced as an exudate following wounding while in guayule, rubber is deposited within the cortical parenchyma and in cells lining the resin duct. In guayule, rubber production is strongly influenced by environmental conditions, e.g., cold temperatures. Yields of latex from guayule are relatively poor, as breeding of commercial varieties for increased production has been complicated by an apomyctic mode of reproduction and difficulties in evaluating latex yields in individual plants. Latex accumulation in guayule is largely dependent upon environmental factors, resulting in highly variable yields and restricting the areas where guayule can be cultivated for latex production. Previous studies of latex production show that latex production is promoted by cold and water restriction in guayule.

Prior attempts to increase latex production in guayule focused on the overexpression of isoprenoid biosynthetic enzymes thought to be rate limiting. This strategy has not been successful to date: overexpression of these enzymes increased metabolic flux into the mevalonate pathway, however latex production was not significantly increased (see, Dong et al., Industrial Crops & Products 46:15-24, 2013.

Additional methods of increasing rubber production in plants by expressing DREB/CBF transcription factors are described in WO2016/161359.

### BRIEF SUMMARY OF THE INVENTION

In one aspect of the invention, provided herein are methods of genetically modifying Asteraceae rubber producing plants, guayule (Parthenium argentatum A. Gray) and Russian dandelion (Taraxacum kok-saghyz), to overexpress one or more of three master regulator transcription factor genes to increase the natural rubber content of guayule (Parthenium argentatum A. Gray). Several technical problems were overcome in identifying master regulator guayule transcription factor genes, in view of the absence of genomics resources and physiological knowledge of guayule, including establishing experimental laboratory growth conditions for guayule that were inductive and repressive for rubber biosynthesis in order to evaluate the endogenous genes.

Latex-producing plants modified in accordance with the invention, e.g., guayule plants, have increased latex production relative to wildtype guayule plants that are not modified to overexpress a guayule transcription factor. Such plants can be used for the extraction of latex.

In certain aspect, provided herein is a method of engineering a rubber-producing plant that is a member of the family Asteraceae to increase latex production, the method comprising: introducing an expression cassette into the rubber-producing plant, wherein the expression cassette comprises a polynucleotide encoding a rubber production transcription factor operably linked to a promoter and further, wherein, the rubber production transcription factor comprises an amino acid sequence having at least 70% identity to SEQ ID NO:2, SEQ ID NO:4, or SEQ ID NO:6; culturing the rubber producing plant under conditions in which the transcription factor is expressed; and selecting a plant that has increased rubber production compared to a counterpart wildtype rubber-producing plant. In some embodiments, the rubber-producing plant is Russian dandelion or guayule. In some embodiments, the transcription factor comprises an amino acid sequence that has at least 75%, at least 80%, at least 85%, at least 90%, or at least 95% identity to SEQ ID NO:2. In some embodiments, the transcription factor comprises an amino acid sequence that has at least 80%, at least 85%, at least 90%, or at least 95% identity to SEQ ID NO:4. In some embodiments, the transcription factor comprises an amino acid sequence that has at least 80%, at least 85%, at least 90%, or at least 95% identity to SEQ ID NO:6. In some embodiments, the transcription factor comprises the amino acid sequence of SEQ ID NO:2, SEQ ID NO:4, or SEQ ID NO:6. In some embodiments, the promoter is a stem-specific promoter. In some embodiments, the promoter is aan inducible promoter.

In a further aspect, provided herein is a plant engineered by the method of the preceiding paragraph, or a progeny of the plant that comprises the expression cassette.

In another aspect, provided herein is a guayule plant that comprises a recombinant expression cassette that comprises a polynucleotide encoding a rubber production transcription factor operably linked to a promoter, wherein the rubber production transcription factor comprises an amino acid sequence having at least 70% identity to SEQ ID NO:2, SEQ ID NO:4, or SEQ ID NO:6. In some embodiments, the transcription factor comprises an amino acid sequence that has at least 75%, at least 80%, at least 85%, at least 90%, or at least 95% identity to SEQ ID NO:2. In some embodiments, the transcription factor comprises an amino acid sequence that has at least 80%, at least 85%, at least 90%, or at least 95% identity to SEQ ID NO:4. In some embodiments, the transcription factor comprises an amino acid sequence that has at least 80%, at least 85%, at least 90%, or at least 95% identity to SEQ ID NO:6. In some embodiments, the transcription factor comprises the amino acid sequence of SEQ ID NO:2, SEQ ID NO:4, or SEQ ID NO:6. In some embodiments, the promoter is a stem-specific promoter or an inducible promoter.

In an additional aspect, provided herein is a method of obtaining latex, the method comprising extracting latex from a plant engineered as described in the preceding paragraphs. In some embodiments, the plant from which latex is extracted is engineered to express a transcription factor that comprises an amino acid sequence that has at least 75%, at least 80%, at least 85%, at least 90%, or at least 95% identity to SEQ ID NO:2. In some embodiments, the transcription factor comprises an amino acid sequence that has at least 80%, at least 85%, at least 90%, or at least 95% identity to SEQ ID NO:4. In some embodiments, the transcription factor comprises an amino acid sequence that has at least 80%, at least 85%, at least 90%, or at least 95% identity to SEQ ID NO:6. In some embodiments, the transcription factor comprises the amino acid sequence of SEQ ID NO:2, SEQ ID NO:4, or SEQ ID NO:6. In some embodiments, the plant from which latex is engineered such that the rubber-producing transcription factor is operably linked to a stem-specific promoter or an inducible promoter.

### BRIEF DESCRIPTION OF THE DRAWINGS

FIG. 1A-1C: Verification of rubber biosynthesis induction in guayule plants by exposure to simulated winter conditions for 2 months. FIGS. 1A and 1B show washed rubber particle extractions from plants grown in simulated summer (Panel 1A) and winter (Panel 1B) conditions. FIG. 1C shows the results of quantitative RT-PCR of rubber biosynthetic enzyme transcripts. *p<0.0, **p<0.001, T-Test.

FIG. 2A-2B: Differential gene expression in guayule transcriptome analysis. FIG. 2A shows a Venn diagram showing the number of differentially expressed genes in common between different tissues. FIG. 2B shows the number of up and down-regulated genes in comparisons of gene expression between each tissue.

FIG. 3: shows that transcirpts encoding SEQ ID NOS:2, 4, and 6 are specifically expressed in rubber-producing, cold-induced stem tissues in guayule. three bars, left to right: TR132611, TR78450, TR113762

### DETAILED DESCRIPTION OF THE INVENTION

### I. Definitions

As used herein, the term “latex production transcription factor” refers to a transcription factor as described herein, i.e., a TR78450, TR113762, or TR132611 transcription factor, or a biologically active variant thereof, that induces expression of one or more genes involved in rubber biosynthesis in guayule.

A “TR78450 transcription factor” as used herein refers to a transcription factor encoded by the nucleic acid sequence of SEQ ID NO:1; and biologically active variant thereof, that has at least 60%, 65%, 70%, 75%, 80%, 85%, 90%, or 95%, or greater, amino acid sequence identity to the amino acid sequence of SEQ ID NO:2 over a region of at least about 100 or 150 amino acids, or over the full-length of the amino acid sequence of SEQ ID NO:2. In some embodiments, a “TR78450 transcription factor” has at least 90% identity; often at least 91%, 92%, 93%, 94%, 95%, 96%, 97%, 98% or 99% amino acid sequence identity to a naturally occurring TR78450 transcription factor from a plant of the Asteraceae, or to a naturally occurring TR78450 transcription factor from a plant of the genus Taraxacum, over the length of the sequence. In some embodiments, a “TR78450 transcription factor” has at least 90% identity; often at least 91%, 92%, 93%, 94%, 95%, 96%, 97%, 98% or 99% amino acid sequence identity to a naturally occurring TR78450 transcription factor from the species Taraxacum kok-saghyz over the length of the sequence. An illustrative TR78450 polypeptide sequence is provided in SEQ ID NO:2.

A “TR78450 transcription factor polynucleotide” as used herein refers to a polynucleotide that encodes a TR78450 transcription factor polypeptide as described in the previous paragraph. A nucleic acid or polynucleotide that encodes a TR78450 transcription factor refers to a gene, pre-mRNA, mRNA, and the like, including nucleic acids encoding variants, alleles, and fragments. An illustrative nucleic acid sequences encoding a TR78450 transcription factor is provided in SEQ ID NO:1. In some embodiments, a “TR78450 transcription factor polynucleotide” has at least 50%, at least 55%, at least 60%, or at least 70%, at least 75%, at least 80%, at least 85%, at least 90%, or greater nucleic acid sequence identity to a naturally occurring TR78450 transcription factor nucleotide sequence, e.g., an mRNA or cDNA generated from the mRNA, from a plant of the Asteraceae, or to a naturally occurring TR78450 transcription factor polynucleotide from a plant of the genus Taraxacum, over the length of the sequence. In some embodiments, a “TR78450 transcription factor polynucleotide” has at least 50%, at least 55%, at least 60%, or at least 70%, at least 75%, at least 80%, at least 85%, at least 90%, or greater nucleic acid sequence identity to a naturally occurring TR78450 transcription factor nucleotide sequence, e.g., an mRNA or cDNA generated from the mRNA, from a plant of the species Taraxacum kok-saghyz over the length of the sequence. In some embodiments, a TR78450 transcription factor polynucleotide at least 50%, or at least 60%, 65%, 70%, 75%, 80%, 85%, 90%, or 95%, or greater, nucleic acid sequence identity to the nucleotide sequence of SEQ ID NO:1.

A “TR113762 transcription factor” as used herein refers to a transcription factor encoded by the nucleic acid sequence of SEQ ID NO:3; and biologically active variant thereof, that has at least 60%, 65%, 70%, 75%, 80%, 85%, 90%, or 95%, or greater, amino acid sequence identity to the amino acid sequence of SEQ ID NO:4 over a region of at least about 100 or 150 amino acids, or over the full-length of the amino acid sequence of SEQ ID NO:4. In some embodiments, a “TR113762 transcription factor” has at least 90% identity; often at least 91%, 92%, 93%, 94%, 95%, 96%, 97%, 98% or 99% amino acid sequence identity to a naturally occurring TR113762 transcription factor from a plant of the Asteraceae, or to a naturally occurring TR113762 transcription factor from a plant of the genus Taraxacum, over the length of the sequence. In some embodiments, a “TR113762 transcription factor” has at least 90% identity; often at least 91%, 92%, 93%, 94%, 95%, 96%, 97%, 98% or 99% amino acid sequence identity to a naturally occurring TR113762 transcription factor from the species Taraxacum kok-saghyz over the length of the sequence. An illustrative TR113762 polypeptide sequence is provided in SEQ ID NO:4.

A “TR113762 transcription factor polynucleotide” as used herein refers to a polynucleotide that encodes a TR113762 transcription factor polypeptide as described in the previous paragraph. A nucleic acid or polynucleotide that encodes a TR113762 transcription factor refers to a gene, pre-mRNA, mRNA, and the like, including nucleic acids encoding variants, alleles, and fragments. An illustrative nucleic acid sequences encoding a TR113762 transcription factor is provided in SEQ ID NO:3. In some embodiments, a “TR113762 transcription factor polynucleotide” has at least 50%, at least 55%, at least 60%, or at least 70%, at least 75%, at least 80%, at least 85%, at least 90%, or greater nucleic acid sequence identity to a naturally occurring TR113762 transcription factor nucleotide sequence, e.g., an mRNA or cDNA generated from the mRNA, from a plant of the Asteraceae, or to a naturally occurring TR113762 transcription factor polynucleotide from a plant of the genus Taraxacum, over the length of the sequence. In some embodiments, a “TR113762 transcription factor polynucleotide” has at least 50%, at least 55%, at least 60%, or at least 70%, at least 75%, at least 80%, at least 85%, at least 90%, or greater nucleic acid sequence identity to a naturally occurring TR113762 transcription factor nucleotide sequence, e.g., an mRNA or cDNA generated from the mRNA, from a plant of the species Taraxacum kok-saghyz over the length of the sequence. In some embodiments, a TR113762 transcription factor polynucleotide has at least 50%, or at least 60%, 65%, 70%, 75%, 80%, 85%, 90%, or 95%, or greater, nucleic acid sequence identity to the nucleotide sequence of SEQ ID NO:3.

A “TR132611 transcription factor” as used herein refers to a transcription factor encoded by the nucleic acid sequence of SEQ ID NO:5; and biologically active variant thereof, that has at least 60%, 65%, 70%, 75%, 80%, 85%, 90%, or 95%, or greater, amino acid sequence identity to the amino acid sequence of SEQ ID NO:6 over a region of at least about 100 or 150 amino acids, or over the full-length of the amino acid sequence of SEQ ID NO:6. In some embodiments, a “TR132611 transcription factor” has at least 90% identity; often at least 91%, 92%, 93%, 94%, 95%, 96%, 97%, 98% or 99% amino acid sequence identity to a naturally occurring TR132611 transcription factor from a plant of the Asteraceae, or to a naturally occurring TR132611 transcription factor from a plant of the genus Taraxacum, over the length of the sequence. In some embodiments, a “TR132611 transcription factor” has at least 90% identity; often at least 91%, 92%, 93%, 94%, 95%, 96%, 97%, 98% or 99% amino acid sequence identity to a naturally occurring TR132611 transcription factor from the species Taraxacum kok-saghyz over the length of the sequence. An illustrative TR113762 polypeptide sequence is provided in SEQ ID NO:6.

A “TR132611 transcription factor polynucleotide” as used herein refers to a polynucleotide that encodes a TR132611 transcription factor polypeptide as described in the previous paragraph. A nucleic acid or polynucleotide that encodes a TR132611 refers to a gene, pre-mRNA, mRNA, and the like, including nucleic acids encoding variants, alleles, and fragments. An illustrative nucleic acid sequence encoding a TR132611 transcription factor is provided in SEQ ID NO:5. In some embodiments, a “TR132611 transcription factor polynucleotide” has at least 50%, at least 55%, at least 60%, or at least 70%, at least 75%, at least 80%, at least 85%, at least 90%, or greater nucleic acid sequence identity to a naturally occurring TR132611 transcription factor nucleotide sequence, e.g., an mRNA or cDNA generated from the mRNA, from a plant of the Asteraceae, or to a naturally occurring TR132611 transcription factor polynucleotide from a plant of the genus Taraxacum, over the length of the sequence. In some embodiments, a “TR132611 transcription factor polynucleotide” has at least 50%, at least 55%, at least 60%, or at least 70%, at least 75%, at least 80%, at least 85%, at least 90%, or greater nucleic acid sequence identity to a naturally occurring TR132611 transcription factor nucleotide sequence, e.g., an mRNA or cDNA generated from the mRNA, from a plant of the species Taraxacum kok-saghyz over the length of the sequence. In some embodiments, a TR132611 transcription factor polynucleotide has at at least 50%, or at least 60%, 65%, 70%, 75%, 80%, 85%, 90%, or 95%, or greater, nucleic acid sequence identity to the nucleotide sequence of SEQ ID NO:5.

The terms “numbered with reference to”, or “corresponding to,” or “determined with reference to” when used in the context of the numbering of a given amino acid or polynucleotide sequence, refers to the numbering of the residues of a specified reference sequence when the given amino acid or polynucleotide sequence is compared to the reference sequence. For example, a position of a variant TR78450 polypeptide sequence “corresponds to” a position with reference to polypeptide sequence SEQ ID NO:2 when the variant polypeptide is aligned with the reference polypeptide sequence in a maximal alignment.

The terms “wild type”, “native”, and “naturally occurring” with respect to a latex production transcription factor refers to a transcription factor that has a sequence that occurs in nature.

The term “downstream target,” when used in the context of a downstream target of a latex production transcription factor that regulates a component of a latex production pathway refers to a gene or protein whose expression is directly or indirectly regulated by the transcription factor.

The term “overexpress” or “overexpression” of a latex production transcription factor refers to an increase in the amount of the transcription factor and/or an RNA encoding the transcription factor that is produced in a plant genetically modified to express the plant, e.g., a guayule plant or Asteraceae plant such as a Russian dandelion. In typical embodiments, an expression construct encoding the transcription factor polypeptide has been introduced into the plant. An increased level of expression is typically at least 5%, or at least 10%, 20%, 30%, 40%, 50%, 60%, 70%, 80%, 90%, 100%, or greater, compared to the counterpart unmodified wildtype plant, under non-stress conditions. The unmodified plant need not express the polypeptide. Thus, the term “overexpression” also includes embodiments in which a polypeptide is expressed in a plant that does not natively express the polypeptide. For example, a Russian dandelion “overexpresses” a transcription factor having a sequence of SEQ ID NO:2, 4, or 6, when the transcription factor is expressed in the plant. The Russian dandelion plant need to natively express SEQ ID NO:2, 4, or 6. Increased expression of a polypeptide can be assessed by any number of assays, including, but not limited to, measuring the level of RNA transcribed from the gene encoding the polypeptide, the level of polypeptide, and/or the level of polypeptide activity.

The terms “increased latex production” refers to an increase in the amount of latex produced by a plant, e.g., a guayule plant, genetically modified to overexpress a latex production transcription factor in comparison to the wildtype plant or corresponding control plant of the same line that has not been genetically modified to overexpress the latex production transcription factor. A guayule plant with increased latex production typically produces at last least 10%, 20%, 30%, 40%, 50%, 60%, 70%, 80%, or 90% or greater compared to a wildtype plant grown under non-stress conditions.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer. These cis-acting sequences typically interact with proteins or other biomolecules to carry out (turn on/off, regulate, modulate, etc.) gene transcription. Promoters are located 5′ to the transcribed gene, and as used herein, typically include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is referred to by the name of the gene for which it naturally regulates expression. Reference to a promoter by name includes a wildtype, native promoter as well as variants of the promoter that retain the ability to induce expression. Reference to a promoter by name is not restricted to a particular plants species, but also encompasses a promoter from a corresponding gene in other plant species.

A “constitutive promoter” in the context of this invention refers to a promoter that is capable of initiating transcription in nearly all cell types, whereas a “cell type-specific promoter” or “tissue-specific promoter” initiates transcription only in one or a few particular cell types or groups of cells forming a tissue. In some embodiments, a promoter is tissue—specific if the transcription levels initiated by the promoter in a tissue are at least 2-fold, 3-fold, 4-fold, 5-fold, or 10-fold, or higher as compared to the transcription levels initiated by the promoter in an unrelated tissue. In some embodiments, the promoter is a “strong” tissue-specific promoter that initiates transcription levels that result in at least 5-fold or at least 10-fold increased expression of a transcript compared to another tissue.

A polynucleotide is “heterologous” to an organism or a second polynucleotide sequence if it originates from a foreign species, or, if from the same species, is modified from its original form or is in a non-naturally occurring structural relationship. For example, when a polynucleotide encoding a polypeptide sequence is said to be operably linked to a heterologous promoter, it can mean that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

As used herein, “recombinant” used in reference to a cell or vector, refers to a cell or vector that has been modified by the introduction of a heterologous nucleic acid sequence or that the cell is derived from a cell so modified. Thus, for example, recombinant cells express genes that are not found in identical form within the native (naturally occurring, non-recombinant) form of the cell or express native genes that are otherwise abnormally expressed, under expressed or not expressed at all as a result of deliberate human intervention. Thus, “recombinant” or “engineered” or “non-naturally occurring” when used with reference to a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. The term encompasses progeny of cells that have been manipulated using recombinant techniques.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “expression cassette” or “DNA construct” or “expression construct” refers to a nucleic acid construct that, when introduced into a host cell, results in transcription and/or translation of an RNA or polypeptide, respectively. Antisense or sense constructs that are not or cannot be translated are expressly included by this definition. Expression constructs can include multiple elements, e.g., a promoter, an enhancer, a transcription terminator, an origin of replication, a chromosomal integration sequence, 5′ and 3′ untranslated regions, or an intronic sequence, which are involved in transcriptional regulation, and the like. In the case of both expression of transgenes and suppression of endogenous genes one of skill will recognize that the inserted polynucleotide sequence need not be identical, but may be only substantially identical to a sequence of the gene from which it was derived. One example of an expression cassette is a polynucleotide construct that comprises a polynucleotide sequence encoding a latex production transcription factor operably linked to a heterologous promoter. In some embodiments, an expression cassette comprises a polynucleotide sequence encoding a latex production transcription factor that is targeted to a position in a plant genome such that expression of the polynucleotide sequence is driven by a promoter that is present in the plant

The term “plant” as used herein can refer to a whole plant or part of a plant, e.g., seeds, and includes plants of a variety of ploidy levels, including aneuploid, polyploid, diploid and haploid. The term “plant part,” as used herein, refers to shoot vegetative organs and/or structures (e.g., leaves, stems and tubers), branches, roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, and plant tissue (e.g., vascular tissue, ground tissue, and the like), as well as individual plant cells, groups of plant cells (e.g., cultured plant cells), protoplasts, plant extracts, and seeds. In some embodiments of the present invention, guayule plants are genetically modified. In other embodiments, Russian dandelion plants are genetically modified.

### Latex Production Transcription Factors

In the present invention, a guayule plant, or alternative plant such as a Russian dandelion plant, is genetically modified to overexpress a latex transcription factor. In some embodiments, the transcription factor comprises a sequence of any one of SEQ ID NOS:2, 4, o 6, or a variant that comprises at least 70% identity, typically at least 75% identity, at least 80% identity, at least 85% identity, at least 90% identity, at least 95% identity, or at least 96%, at least 97%, at least 98%, or at least 99% to any one of SEQ ID NO:2, 4, or 6.

Methods and computer programs for the alignment are well known in the art. The term “identity” or “homology” as used here refers to the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software. Examples include BLAST or BLAST 2.0 with default parameters.

For sequence comparison of polypeptides, typically one amino acid sequence acts as a reference sequence, to which a candidate sequence is compared. As indicated above, alignment can be performed using various methods available to one of skill in the art, e.g., visual alignment or using publicly available software using known algorithms to achieve maximal alignment. Such programs include the BLAST programs or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

In some embodiments, a guayule plant or alternative plant such as a Russian dandelion plant, is genetically modified to overexpress a TR78450 transcription factor or variant as described herein. TR78450 is a member of the CBF/DREB family of transcription factors. Many Asteraceae family members that produce latex have transcription factors that have about 70% to about 85% identity to SEQ ID NO:2. An illustrative ortholog is the native sunflower sequence available under accession number XP_022014201.1, which has over 80% identity to SEQ ID NO:2. In some embodiments, a plant is genetically modified to express a TR78450 transcription factor has at least 90%, 91%, 92%, 93%, 94%, 95%, 96%, 97%, 98%, or 99% identity to such a native transcription factor, i.e., that naturally occurs in an Asteraceae family member that produces latex, such as the sunflower polypeptide sequence available under accession number XP_022014201.1.

In some embodiments, a guayule plant or alternative plant such as a Russian dandelion plant, is genetically modified to overexpress a TR113762 transcription factor or variant as described herein. TR113762 is a member of the basic leucine zipper-like family of transcription factors. A corresponding sequence in sunflower has about 85% identity to SEQ ID NO:4. The polypeptide sequence of the sunflower ortholog is available under GenBank accession number XP 021998452.1. In some embodiments, a plant is genetically modified to express a TR113762 transcription factor has at least 90%, 91%, 92%, 93%, 94%, 95%, 96%, 97%, 98%, or 99% identity to the native transcription factor polypeptide sequence available under accession number XP_021998452.1.

In some embodiments, a guayule plant or alternative plant such as a Russian dandelion plant, is genetically modified to overexpress a TR132611 transcription factor or variant as described herein. TR132611 is a member of the LBD41 Lob domain-containing family of transcription factors. A corresponding sequence in sunflower has about 85% identity to SEQ ID NO:6. The polypeptide sequence of the sunflower ortholog is available under GenBank accession number XP_022009468.1 In some embodiments, a plant is genetically modified to express a R132611 transcription factor has at least 90%, 91%, 92%, 93%, 94%, 95%, 96%, 97%, 98%, or 99% identity to the native transcription factor polypeptide sequence available under accession number XP_022009468.1.

### Latex Production Transcription Factors Nucleic Acid Sequences

The invention employs various routine recombinant nucleic acid techniques. Generally, the nomenclature and the laboratory procedures in recombinant DNA technology described below are those well-known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2009, supplements through 2017).

In some embodiments of the present invention, a guayule plant, or an alternative plant such as a Russian dandelion plant, is genetically modified to constitutively express a latex production transcription factor TR78450, TR113762, or TR132611. In other embodiments, the plant may be genetically modified to express the transcription factor under the control of a tissue-specific promoter, e.g., a leaf promoter, or an inducible promoter, such as an ethanol-inducible promoter, to increase latex production.

Isolation or generation of TR78450, TR113762, or TR132611 polynucleotides can be accomplished by a number of well-known techniques. In some embodiments, oligonucleotide probes based on the sequences disclosed here can be used to identify the desired polynucleotide in a cDNA or genomic DNA library from a desired plant species. In typical embodiments, the nucleic acids of interest can be amplified from nucleic acid samples using routine amplification techniques. For instance, PCR may be used to amplify the sequences of the genes directly from mRNA, from cDNA, from genomic libraries or cDNA libraries. PCR and other in vitro amplification methods may also be useful for other purposes, e.g., nucleic acid sequencing, to obtain a desired fragment of a polynucleotide of interest. In the context of the present invention, a TR78450, TR113762, or TR132611 gene refers to a polynucleotide sequence that encodes the TR78450, TR113762, or TR132611 polypeptide. The gene may comprise a cDNA sequence or genomic DNA, sequence.

### Preparation of Recombinant Vectors

To use isolated sequences in the above techniques, recombinant DNA constructs suitable for transformation of guayule cells, or alternative cells such as Russian dandelion cells, are prepared. Techniques for preparing such constructs are well known and described in the technical and scientific literature. For example, a DNA sequence encoding a TR78450, TR113762, or TR132611 transcription factor can be combined with transcriptional and other regulatory sequences which will direct the transcription of the sequence from the gene in the intended cells, e.g., guayule stem cells. In some embodiments, an expression vector that comprises an expression cassette that comprises the TR78450, TR113762, or TR132611 gene further comprises a promoter operably linked to the TR78450, TR113762, or TR132611 gene. In other embodiments, a promoter and/or other regulatory elements that direct transcription of the TR78450, TR113762, or TR132611 gene are endogenous to the plant and an expression cassette comprising the TR78450, TR113762, or TR132611 gene is introduced, e.g., by homologous recombination, such that the heterologous TR78450, TR113762, or TR132611 gene is operably linked to an endogenous promoter and is expression driven by the endogenous promoter. In some embodiments, a promoter that drives expression of the TR78450, TR113762, or TR132611 gene may be a promoter of a gene involved in latex production in guayule. Any number of promoters may be used to drive expression of the TR78450, TR113762, or TR132611 gene, including either constitutive or inducible, or tissue-specific promoters.

### Constitutive Promoters

A promoter, or an active fragment thereof, can be employed which will direct expression of a nucleic acid encoding a fusion protein of the invention, in all or most transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless, Arch. Virol. 142:183-191, 1997); the 1′- or 2′-promoter derived from T-DNA of Agrobacterium tumefaciens (see, e.g., Mengiste supra (1997); O'Grady, Plant Mol. Biol. 29:99-108, 1995); the promoter of the tobacco mosaic virus; the promoter of Figwort mosaic virus (see, e.g., Maiti, Transgenic Res. 6:143-156, 1997); ubiquitin promoters, actin promoters, such as the Arabidopsis actin gene promoter (see, e.g., Huang, Plant Mol. Biol. 33:125-139, 1997); alcohol dehydrogenase (Adh) gene promoters (see, e.g., Millar, Plant Mol. Biol. 31:897-904, 1996); ACT11 from Arabidopsis (Huang et al., Plant Mol. Biol. 33:125-139, 1996), Cat3 from Arabidopsis (GenBank No. U43147, Zhong et al., Mol. Gen. Genet. 251:196-203, 1996), the gene encoding stearoyl-acyl carrier protein desaturase from Brassica napus (Genbank No. X74782, Solocombe et al., Plant Physiol. 104:1167-1176, 1994), GPc1 from maize (GenBank No. X15596, Martinez et al., J Mol. Biol. 208:551-565, 1989), Gpc2 from maize (GenBank No. U45855, Manjunath et al., Plant Mol. Biol. 33:97-112, 1997), other transcription initiation regions from various plant genes known to those of skill. See also Holtorf, “Comparison of different constitutive and inducible promoters for the overexpression of transgenes in Arabidopsis thaliana,” Plant Mol. Biol. 29:637-646, 1995).

### Tissue-Specific Promoters

In some embodiments, a plant promoter to direct expression of TR78450, TR113762, or TR132611 gene in a specific tissue is employed (tissue-specific promoters). Tissue specific promoters are transcriptional control elements that are only active in particular cells or tissues at specific times during plant development.

Tissue-specific promoters include promoters that initiate transcription only (or primarily only) in certain tissues, such as vegetative tissues, cell walls, roots or leaves. A variety of promoters specifically active in vegetative tissues, such as leaves, stems, and roots are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarin promoter of the gene encoding a globulin from a major taro (Colocasia esculenta L. Schott) corm protein family, tarn (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are kn1-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

Other examples of promoters are secondary cell wall promoters such as IRX1, IRX3, IRX5, IRX8, IRX9, IRX14, IRX7, IRX10, GAUT13, or GAUT14 promoters.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

### Inducible Promoters

In some embodiments, an inducible promoter is used. Examples of inducible promoters include plant promoters that are inducible upon exposure to plant hormones, such as auxins. For example, the invention can use the auxin-response elements E1 promoter fragment (AuxREs) in the soybean (Glycine max L.) (Liu, Plant Physiol. 115:397-407, 1997); the auxin-responsive Arabidopsis GST6 promoter (also responsive to salicylic acid and hydrogen peroxide) (Chen, Plant J. 10: 955-966, 1996); the auxin-inducible parC promoter from tobacco (Sakai, 37:906-913, 1996); or a plant biotin response element (Streit, Mol. Plant Microbe Interact. 10:933-937, 1997). Other examples of useful promoters include alcohol-inducible promoters, e.g., an ethanol inducible promoter.

Plant promoters inducible upon exposure to chemicals reagents that may be applied to the plant, such as herbicides or antibiotics, are also useful for expressing TR78450, TR113762, or TR132611 gene in accordance with the invention. For example, the maize In2-2 promoter, activated by benzenesulfonamide herbicide safeners, can be used (De Veylder, Plant Cell Physiol. 38:568-577, 19997); application of different herbicide safeners induces distinct gene expression patterns, including expression in the root, hydathodes, and the shoot apical meristem. A TR78450, TR113762, or TR132611 coding sequence can also be under the control of, e.g., a tetracycline-inducible promoter, such as described with transgenic tobacco plants containing the Avena sativa L. (oat) arginine decarboxylase gene (Masgrau, Plant J. 11:465-473, 1997); or, a salicylic acid-responsive element (Stange, Plant J. 11:1315-1324, 1997; Uknes et al., Plant Cell 5:159-169, 1993); Bi et al., Plant J 8:235-245, 1995).

Further examples of inducible regulatory elements include copper-inducible regulatory elements (Mett et al., Proc. Natl. Acad. Sci. USA 90:4567-4571, 1993); Furst et al., Cell 55:705-717, 1988); tetracycline and chlor-tetracycline-inducible regulatory elements (Gatz et al., Plant J. 2:397-404, 1992); Roder et al., Mol. Gen. Genet. 243:32-38, 1994); Gatz, Meth. Cell Biol. 50:411-424, 1995); ecdysone inducible regulatory elements (Christopherson et al., Proc. Natl. Acad. Sci. USA 89:6314-6318, 1992; Kreutzweiser et al., Ecotoxicol. Environ. Safety 28:14-24, 1994); heat shock inducible regulatory elements (Takahashi et al., Plant Physiol. 99:383-390, 1992; Yabe et al., Plant Cell Physiol. 35:1207-1219, 1994; Ueda et al., Mol. Gen. Genet. 250:533-539, 1996); and lac operon elements, which are used in combination with a constitutively expressed lac repressor to confer, for example, IPTG-inducible expression (Wilde et al., EMBO J. 11:1251-1259, 1992). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

### Additional Promoters

In some embodiments, endogenous promoters from genes in the latex biosynthesis pathway can be used to drive expression of transcription factors activating the biosynthesis of rubber, producing an artificial positive feedback loop to drive latex production strongly once it has been naturally induced. Promoters of known rubber biosynthesis genes previously identified in guayule and known to be highly expressed in latex producing tissue, such as SRPP, farnesyl-phosphate synthase and allene oxide synthase can be used for this purpose (see, e.g., Ponciano et al., Phytochemistry 79:57-66, 2012, which is incorporated by reference). Additional promoters from Rubber Elongation Factor, hydroxymethylglutaryl CoA synthase, Cis-prenyltransferase, Cis-prenyl-transferase Like, and allene oxide synthase can also be employed. Thus, for example, an expression cassette may comprise a polynucleotide encoding a rubber-producing transcription factor of SEQ ID NO:2, SEQ ID NO:4, or SEQ ID NO:6, or a variant thereof having at least 70% identity to SEQ ID NO:2, SEQ ID NO:4, or SEQ ID NO:6 operably linked to a promoter, e.g., a native promoter, of a known rubber biosynthesis gene, such as promoters from Rubber Elongation Factor, hydroxymethylglutaryl CoA synthase, Cis-prenyltransferase, Cis-prenyl-transferase Like, and allene oxide synthase. In some embodiments, such promoters extend to 200 or 500 base pairs upstream of the transcription initiation site. In some embodiments, the promoters are from 100 to 500 base pairs in length. In some embodiments, the promoters are from 200 to 500 base pairs in length.

### Additional Embodiments for Expressing a TR78450, TR113762, or TR132611 Transcription Factor

In another embodiment, a TR78450, TR113762, or TR132611 polynucleotide is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai, Proc. Natl. Acad. Sci. USA 92:1679-1683, 1995); the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer, Plant Mol. Biol. 31:1129-1139, 1996).

A vector comprising TR78450, TR113762, or TR132611 nucleic acid sequences will typically comprise a marker gene that confers a selectable phenotype on the cell to which it is introduced. Such markers are known. For example, the marker may encode antibiotic resistance, such as resistance to kanamycin, G418, bleomycin, hygromycin, and the like.

Additional sequence modifications may be made that are also known to enhance gene expression in a plant. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence may also be modified to avoid predicted hairpin secondary mRNA structures.

As an example illustrating generation of a construct encoding a rubber master regulatory transcription factor for expression in guayule, primers are designed to amplify a polynucleotide encoding a latex production-regulating transcription factor, e.g., a TR78450, TR113762, or TR132611 transcription factor comprising an amino acid sequence of SEQ ID NO:2, 4, or 6, into an Agrobacterium binary vector for plant transformation and protein expression, such as pCAMBIA2300. Vectors are constructed to drive expression of the transcription factors in guayule, e.g., using a constitutive promoter such as the CaMV 35s or a ubiquitin or rubisco small subunit promoter.

### Production of Transgenic Plants

As detailed herein, the present invention provides for transgenic plants, e.g., guayule plants, comprising recombinant expression cassettes for expressing a TR78450, TR113762, or TR132611 transcription factor. It should be recognized that the term “transgenic plants” as used here encompasses the plant or plant cell in which the expression cassette is introduced as well as progeny of such plants or plant cells that contain the expression cassette, including the progeny that have the expression cassette stably integrated in a chromosome.

Once an expression cassette comprising a polynucleotide encoding a TR78450, TR113762, or TR132611 transcription factor has been constructed, standard techniques may be used to introduce the polynucleotide into a plant in order to modify gene expression. See, e.g., protocols described in Ammirato et al. (1984) Handbook of Plant Cell Culture—Crop Species. Macmillan Publ. Co. Shimamoto et al. (1989) Nature 338:274-276; Fromm et al. (1990) Bio/Technology 8:833-839; and Vasil et al. (1990) Bio/Technology 8:429-434.

Transformation and regeneration of plants is known in the art, and the selection of the most appropriate transformation technique will be determined by the practitioner. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence. Examples of these methods in various plants include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

In one embodiment, a TR78450, TR113762, or TR132611 construct in accordance with the invention can be transformed into guayule using an Agrobacterium co-cultivation technique (see, e.g., Dong et al., Industrial Crops & Products 46:15-24, 2013 and Dong et al., Plant Cell Rep 25:26-34, 2006). In brief, portions of guayule leaf are co-cultivated with Agrobacterium tumeficiens strains carrying the construct of interest on a binary vector for plant transformation. After co-cultivation with the agrobacterium, undifferentiated callus tissue is cultivated from the leaves and transgenic calli are selected. Transgenic plants are then regenerated from the callus tissue. For example, binary vectors carrying Kanamycin resistance or phosphomannose-isomerase genes for selection of transgenic plants can be used for plant transformation with latex production regulating constructs. For phosphomannose isomerase selection, transgenic calli can be selected by providing 20 g/l mannose as the only carbon source, e.g., as described by Wang et al., Plant Cell Rep 19:654-660, 2000. Transgenic calli can be transferred to regeneration medium for rooting then transferred to grow on soil. Transgenic plants can then be propagated by cuttings prior to testing for latex accumulation with and without an inducer, if an inducible promoter is used, such as an ethanol-inducible promoter, or cold induction of latex production by previously described methods such as accelerated solvent extraction.

Transformed plant cells derived by any of the above transformation techniques can be cultured to regenerate a whole plant that possesses the transformed genotype and thus the desired phenotype. Such regeneration techniques may involve manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally, e.g., in Klee et al. Ann. Rev. of Plant Phys. 38:467-486, 1987.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

A guayule plant that is genetically modified to express a TR78450, TR113762, or TR132611 polypeptide can be identified using any known assay, including analysis of RNA, protein, or latex content compared to a wildtype guayule plant. With respect to this aspect of the invention, the plants have enhanced latex levels.

A guayule plant, or alternative plant such as a Russian dandelion, genetically modified in accordance with the invention can be used to obtain latex. Methods of extracting latex from guayule, or other plants, are known in the art. For example, rubber is found primarily in the bark and is released during processing. Plant material comprising bark, e.g., the whole plant or branches, are homogenized in an aqueous extraction medium. The rubber particles obtained from the parenchyma cells of the guayule plant are thereby released into the solution to create an aqueous suspension comprising the particles. The rubber particles, which have a specific gravity of slightly less than 1, can then be purified from the homogenate using a series of centrifugation steps and/or flotation with creaming agents. This process results in natural rubber latex with very little remaining cytoplasmic or soluble protein components. Examples of methods of extracting latex are provided in e.g., WO2007081376; U.S. Patent Application Publication Nos. 20110021743, 20070265408, and 20060106183; U.S. Pat. Nos. 5,717,050, 5,580,942, and 6,054,525, each of which are incorporated by referenced. Purification of latex is also described in Cornish K. and J. L. Brichta. 2002. Purification of hypoallergenic latex from guayule. p. 226-233, 2002, In: J. Janick and A. Whipkey (eds.), Trends in new crops and new uses. ASHS Press, Alexandria, Va., also incorporated by reference, and references cited therein.

### EXAMPLES

### Induction of Rubber Biosynthesis

Rubber biosynthesis in guayule occurs when the shrub experiences low nighttime temperatures below 5-7° C. during the winter in the cortical parenchyma of stems. Artificial exposure to low temperatures induces rubber accumulation in guayule in plants older than approximately 120 days (Bonner, J. Effects of temperature on rubber accumulation by the guayule plant. Botanical Gazette (1943)). Exposure to low, non-freezing night temperatures in the lab induces rubber biosynthetic activity in guayule (Goss, et al., Plant Physiology 74, 534-537 (1984)). The enzymatic rates of 3-hydroxy-3-methylglutaryl-CoA reductase and rubber transferase enzymes are also seasonally induced (Benedict, et al., Industrial Crops and Products 61, 176-179 (2014)). To study changes in gene expression associated with the production of rubber we sought to establish inductive and repressive growth conditions for guayule rubber biosynthesis in the lab and to validate that changes in the expression of genes associated with rubber biosynthesis were induced.

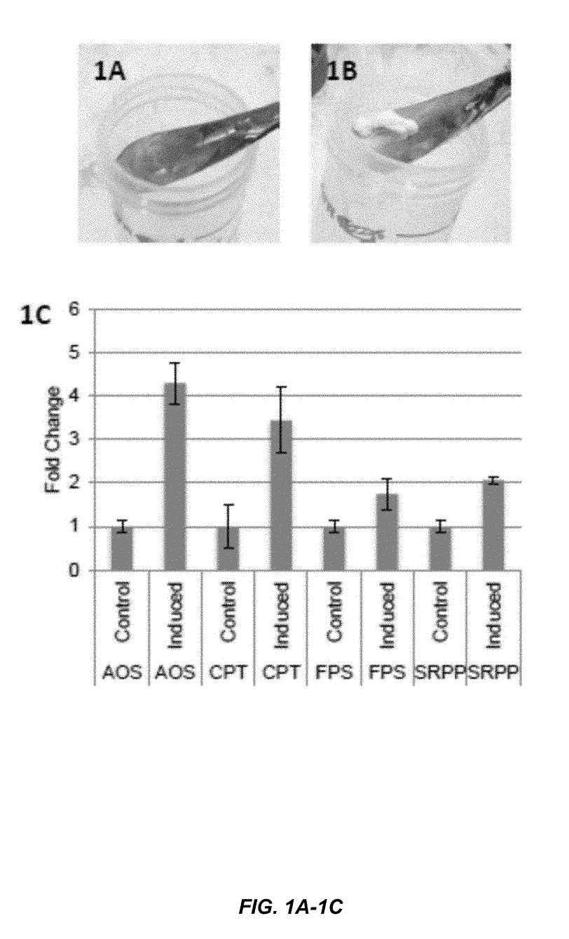

Summer and winter temperature regimes in guayule's native environment were simulated with parameters in concordance with previous inductions of rubber biosynthesis in guayule. Summer conditions were simulated in growth chambers with a 16 hour day with 25° C. daytime and 15° C. nighttime temperatures and winter conditions with an 11 hour day with 25° C. daytime and 5° C. nighttime temperatures. To validate the induction of rubber biosynthesis, we extracted rubber particles from guayule plants exposed to simulated winter and summer conditions for six weeks (FIGS. 1A & 1B). Large amounts of rubber were present in induced plants, forming a thick mat of congealed rubber following centrifugation. Little rubber was present in plants grown in simulated summer conditions. The expression levels of genes thought to be involved in the biosynthesis of rubber, AOS (Allene Oxide Synthase) and CPT (Cis Prenyl-Transferase), have been shown to be weakly correlated with the induction of rubber biosynthesis by winter conditions in guayule (Ponciano et al. Phytochemistry 79, 57-66 (2012)). To validate that these genes were induced in our rubber producing plants, we tested the relative expression of previously identified genes thought to be involved in guayule rubber biosynthesis in stems of plants exposed to simulated summer and winter conditions using real-time RT-PCR. As expected, significant up-regulation of AOS, CPT and FPS transcripts was detected in plants grown in winter conditions (FIG. 1B). We also detected significant induction of the SRPP (small rubber particle protein) transcript, which has not previously been shown to be responsive to low temperatures. We thus concluded that simulated winter conditions were capable of inducing rubber biosynthesis in guayule and that this induction elicited changes in the expression of rubber biosynthetic enzymes.

### RNAseq Analysis and Transcriptome Assembly

To characterize global changes in gene expression in rubber producing tissues and to identify genes involved in the biosynthesis of rubber we performed an RNAseq analysis of control tissues (leaves) and tissues producing rubber (stems) from control and induced plants. We prepared bar-coded, directional RNA-seq libraries from the stems and leaves of 7 month-old guayule plants that had been exposed in simulated winter or summer conditions for 8 weeks. Three biological replicate sequencing libraries were prepared from each tissue. These libraries were pooled and sequenced on MiSeq and Hiseq Illumina sequencers resulting in 28 million 300 bp paired end reads and 155 million 150 bp paired end reads respectively with an average of 2.4 million MiSeq and 12.9 million HiSeq reads per sample (data not shown). The transcriptome was assembled using the Trinity assembler into 200,074 unigenes with a median length of 456 bp and with fifty percent of the total sequence assembled into contigs of 850 bp or larger. The total assembly represented 149 million bp of sequence. As the plants used in this study are of the AZ-2, tetraploid cultivar, the transcriptome assembly contains up to 4 alleles of each gene given that the plants sequenced were the progeny of a tetraploid single plant selection 5. Many transcripts were assembled into distinct “isoforms” likely representing distinct alleles of individual genes. That the transcriptome assembly likely represents up to four distinct contigs for each gene complicates the data by adding allelic variation between individual plants to the analysis of differential gene expression.

### Differential Gene Expression Analysis

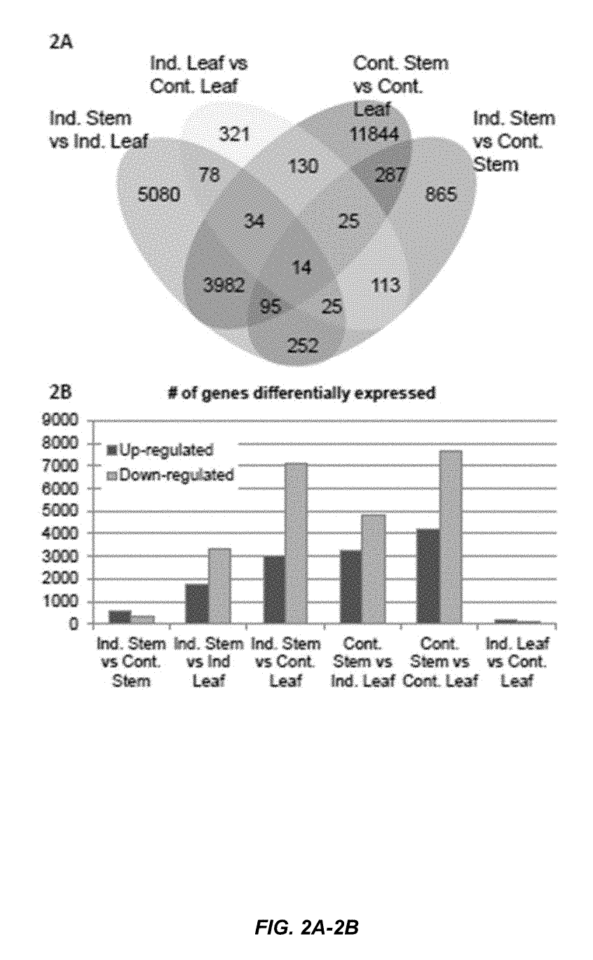

To identify genes involved in guayule rubber biosynthesis, differential gene expression analysis was conducted using the EdgeR package (Robinson et al, Bioinformatics 26:139-140, 2010). Our premise was that genes involved in rubber biosynthesis would be induced under simulated winter conditions in stems, where rubber is produced, but not in leaves where it is not; whereas genes induced by cold but not related to rubber biosynthesis would be induced in both leaves and stems. Differentially expressed contig (16,394) were identified at padj<0.0001 between two or more tissues. In a Venn diagram analysis we analyzed the number of contigs that were differentially expressed between multiple tissues (FIG. 2A). Notably, 3982 transcripts were differentially expressed between stem and leaf tissues in both induced and control plants (FIG. 2A). Only 252 contigs were differentially expressed between induced and control stems as well as between leaves and stems in induced plants; the gene set where we hypothesized that genes involved in the production of rubber would be identified. We also examined the number of contigs up- and down-regulated in each comparison (FIG. 2B). More genes were up-regulated than down-regulated in both stem and leaf tissues following cold treatment. Likewise, cold-induction resulted in the up-regulation of only 205 contigs in leaf tissues and the down-regulation of 116 contigs.

This discrepancy between the number of genes differentially expressed between tissue types and the much smaller number differentially expressed between growth conditions is easily observed in a heat map of hierarchically clustered expression data (data not shown). Hierarchical clustering of differentially expressed contigs shows that most genes exhibited similar expression patterns between tissues in both induced and control plants while a smaller group of genes are specifically expressed in the rubber producing stems of cold-induced plants. Most contigs in this cluster have a background level of expression in the stems of control plants and are more highly expressed in the stems of induced plants.

### Gene Ontology Analysis

To characterize changes in gene expression between tissues, we analyzed the gene ontology term (GOterm) enrichment of differentially expressed contigs with similar expression patterns. We analyzed the GO-terms enriched in each cluster of differentially expressed contigs. In the cluster of contigs most highly expressed in stems of control plants we observed the enrichment of cytoskeletal, lignin catabolic, and photoreceptor activities at p values below 0.005 (data not shown). In the cluster of contigs highly expressed in the rubber producing stems of induced plants contigs encoding hydroxymethylglutaryl-COA reductases, oxidoreductase and terpene synthase activities were enriched at p values below 0.001. Nucleoside metabolic processes were also enriched in the rubber biosynthetic gene set. We also analyzed GO term enrichment in contigs highly expressed in leaf tissues. As expected, most of the GO-terms enriched in the leaf-expressed gene set were for processes related to photosynthesis such as chorophyll binding, ATP metabolic processes and electron transport chain at p values below 0.001. Membrane proteins were overrepresented in this gene set, as were enzymatic activities associated with carbon fixation and electron transport chains.

### Analysis of Highly Expressed and Strongly Induced Genes

To elucidate the metabolic processes occurring in rubber biosynthetic tissues, we analyzed the most highly expressed and most strongly enriched transcripts in induced stem tissues. Highly expressed genes include a protein similar to the ribosome-inactivating lectin that is processed into ricin toxin and various genes involved in stress and defense responses such as defensins,dehydrins, HSPs, metallothionin, COLD-REGULATED 413 and RCI2A. Analysis of the contigs most strongly induced in stem tissue identified many additional genes with associated with responses to stress and the production of terpenoids.

### Identification of Rubber Biosynthetic Enzymes

To characterize the expression of contigs encoding enzymes previously implicated in the production of rubber in guayule we searched the differentially expressed gene set for contigs encoding the small rubber particle protein (SRPP), rubber associated Cis-prenyltransferases (CPT and CPTL), allene oxide synthase (AOS), DXP and Mevalonate pathway enzymes using BLAST then retrieved annotation and expression data for the identified contigs. Through this analysis we identified a set of contigs encoding AOS, CPTs, SRPP, HMGR and GGPS that were strongly expressed in induced stem tissue and with varying background levels of expression in the stems of control plants. A consistent background level of expression between one third and one half the absolute transcript level in induced stem tissue was observed in the stems of control plants. The high standard deviation of transcript levels in this study, likely due to the genetic heterogeneity of the polyploidy plants sequenced, causes comparisons of expression levels between induced and control stems to be above the stringent threshold used for calling statistical significance. The varying levels of expression of these contigs agree well with qPCR data and previous analyses such as that described in Ponciano et al, 2012, supra. Each of the contigs identified in this analysis is significantly up-regulated in induced stems compared to expression levels in leaf tissues and is more highly expressed in induced stems than in control stems but not at a significance level of p<0.0001. The weak, but consistent induction of the SRPP encoding gene and the strong expression of AOS in rubber producing tissue are consistent with previous studies. The presence of many contigs encoding HMGR may also suggest the presence of multiple alleles or tandem duplication and diversification of this gene in guayule and was also observed in GO term enrichment analysis. A previous EST study identified two distinct HMGR encoding ESTs and did not detect cold induction of HMGR (Ponciano et al, 2012, supra) although another study (Benedict et al, 2014, supra) supported that the induced rate of HMGR limits the rate of rubber formation in guayule. Other mevalonate and MEP pathway enzymes were identified in the set of differentially expressed transcripts, however all were more strongly expressed in leaves than in stems and were not significantly up-regulated in stems following cold induction. Homologs of both classes of rubber-biosynthesis associated CPTs were detected, homologs of Arabidopsis cis-prenyltransferase 1 (CPT1) and Nogo-B receptor-like CPTL/LEW1.

### Transcription Factors Expressed in Rubber Biosynthetic Tissues

To identify transcription factor-encoding genes differentially expressed in rubber biosynthetic tissue we searched for contigs with annotated DNA-binding or transcriptional regulation activity in the GO term dataset. Contigus (708) were identified with predicted DNA binding or transcriptional regulation activities. We used hierarchical clustering of the expression of these differentially expressed contigs encoding proteins with DNA-binding or transcriptional regulation activity to identify a cluster with stronger expression in induced stems than in other tissues. This gene set consists of 30 contigs encoding proteins with homology to DNA binding or transcription factors. All but one contig identified through this analysis encode a gene product with homology to previously studied families of transcription factors.). AtGRP2B is a cold-shock domain protein thought to function in the destabilization of RNA secondary structure. Some, but not all, of these contigs are differentially expressed at p<0.0001 between induced stems and all other tissues and many distinct classes of plant transcription factors are represented in the gene set.

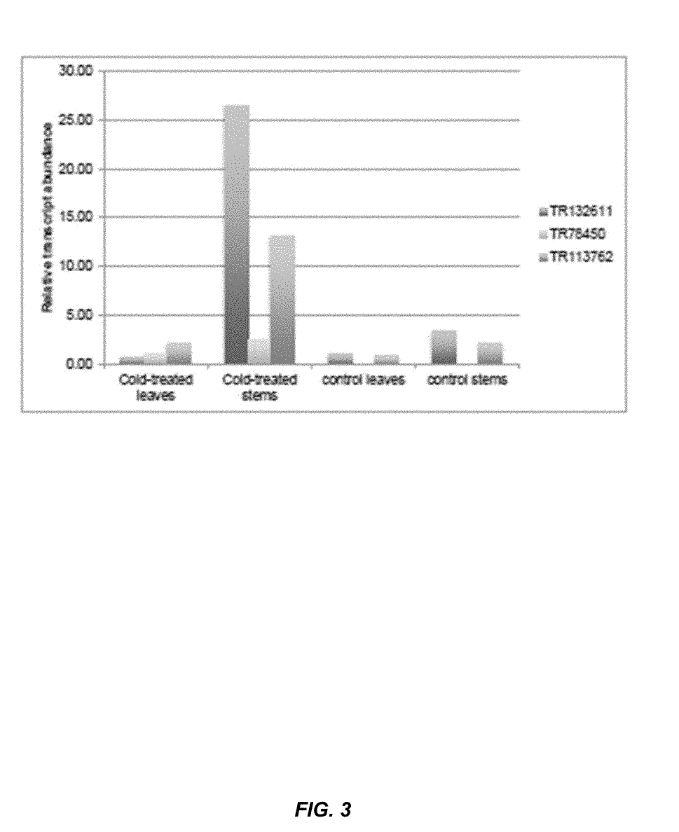

FIG. 3 provides data showing that the transcripts encoding the rubber-producing transcription factors fo SEQ ID NOS:2, 4, and 6 are specfrically expressed in rubber-producing, cold-induced stem tissues in guayule.

### Yeast One-Hybrid Screen

Several technical problems were overcome in this research to overcome the absence of genomics resources for and physiological knowledge of guayule. Establishment of growth conditions in the lab that were inductive and repressive of rubber biosynthesis was first performed. Sequencing and assembly of the guayule transcriptome was technically challenging, as was the differential gene expression analysis leading to the identification of candidate transcription factors. This analysis also yielded a set of candidate rubber biosynthetic enzyme encoding genes. To evaluate the activation of rubber biosynthesis by transcription factors, the promoters of rubber biosynthetic genes had to be sequenced and cloned as was acquired without sequencing the whole guayule genome. To this end we cloned coding sequence of rubber biosynthetic genes acquired in the transcriptome sequencing experiment then used biotinylated RNA prepared from these sequences to enrich a large-insert genome sequencing library for our genes of interest and sequenced the library on the PacBio single-molecule real-time sequencing platform. We were thus able to acquire full-length genomic sequence of rubber biosynthetic genes, including promoter regions.

Transcription factors were analyzed in a yeast-one hybrid experiment to determine if they bind to the promoters of rubber biosynthetic genes. Three of these candidate master regulator transcription factors, TR78450, TR113762, and TR132611, consistently bound to the promoters of multiple rubber biosynthetic genes in yeast one-hybrid experiments supporting that they regulate expression of rubber biosynthesis. A table showing the promoters bound by TR78450, TR113762, or TR132611 is provided below. It was found that the candidate master regulator transcription factors bound to their own promoters and to the promoters of one another, supporting their roles as master regulators of rubber biosynthesis in guayule.

TABLE 1
Promoters bound by the master regulator transcription
factors in yeast one-hybrid experiments:

| Bait promoter | Prey Candidate TF | Prey description |
|---|---|---|
| proricin | C_TR78450 | CBF4, DREB1D C-repeat-binding factor 4 |
| proricin | C_TR113762 | basic leucine zipper-like |
| Retc | C_TR78450 | CBF4, DREB1D C-repeat-binding factor 4 |
| Retc | C_TR113762 | basic leucine zipper-like |
| Retc | C_TR132611 | LBD41 LOB domain-containing protein |
| TerpSyn | C_TR78450 | CBF4, DREB1D C-repeat-binding factor 4 |
| TerpSyn | C_TR113762 | basic leucine zipper-like |
| TerpSyn | C_TR132611 | LBD41 LOB domain-containing protein |
| AOC | C_TR78450 | CBF4, DREB1D C-repeat-binding factor 4 |
| pTR113762 (Leucine zip) | C_TR78450 | CBF4, DREB1D C-repeat-binding factor 4 |
| pTR113762 (Leucine zip) | C_TR113762 | basic leucine zipper-like |
| pTR78450 (CBF) | C_TR132611 | LBD41 LOB domain-containing protein |
| pTR78450 (CBF) | C_TR78450 | CBF4, DREB1D C-repeat-binding factor 4 |

### Materials and Methods:

### Plant Growth

Guayule (Parthenium argentatum Gray) plants of the AZ-2 cultivar were germinated in a greenhouse in Eloy, Ariz. At 4 months of age these plants were transferred to simulated summer conditions in a plant growth chamber (16 h day, 25° C. daytime and 15° C. nighttime temperatures). At 6 months of age plants were transferred to a growth chamber simulating winter conditions (11 h night, 25° C. daytime and 5° C. nighttime temperatures) for induction of rubber biosynthesis. Plants were harvested for analysis at 8 months of age. Plants grown in simulated winter conditions were considered “induced” for rubber biosynthesis. Washed rubber particle extractions were conducted as described by 32.

### Quantitative RT-PCR

RNA was extracted from plant tissues using Trizol (Invitrogen) following the manufacturer's instructions with the exception that following the initial homogenization and incubation in Trizol, insoluble material was pelleted by centrifugation. Following Trizol extraction, RNA samples were treated with DNAase using the Turbo DNAfree kit (Ambion) and further purified using RNeasy column purification (Qiagen). RNA concentration was measured on a Nanodrop spectrophotometer. RNA was reverse transcribed using the Superscript III kit from Invitrogen. Primer sets for qPCR were designed based on Parthenium argentatum sequences previously deposited in genbank as well as EST data from the Compositae genomics project. qPCR was conducted on a Stepone Real-Time PCR system (Applied Biosystems) using SYBR® Select Master Mix (Thermo) according to the manufacturer's instructions. qPCR analysis was conducted in biological duplicate with triplicate technical replication.

### RNA Seq Analysis

Directional, bar-coded illumina sequencing libraries were prepared using the NEBNext® Ultra™ Directional RNA Library Prep Kit for Illumina® (New England Biolabs) according to the manufacturer's instructions with the suggestions for size selection of an average insert size of 300-450 bp. For library preparation, RNA samples were prepared as for qPCR analysis and analyzed using a Bioanalyzer 2100 RNA chip (Agilent Genomics) to evaluate RNA concentration and quality prior to library preparation. Libraries were analyzed on a Bioanalyzer 2100 High Sensitivity DNA assay, pooled at an equimolar ratio. The pooled libraries were then sequenced on Illumina Miseq (2×300 bp) and HiSeq2500 (2×150 bp, Rapid Run mode) platforms.

### Transcriptome Analysis

RNA seq data was quality controlled and adapter sequences removed using Trim Galore (Babraham Bioinformatics group) to eliminate adapter sequence contamination and to trim data below Q30. Miseq and Hiseq reads were pooled for transcriptome assembly. The transcriptome was assembled using Trinity assembler version 2.04 set to minimum kmer coverage of 2 33. The initial transcriptome assembly was filtered using a utility included within the Trinity package to eliminate transcript isoforms with low abundance and low coverage. Differential gene expression analysis was performed using the EdgeR as described in 33. The differentially expressed gene set was selected as contigs with a P value, adjusted for the false-discovery rate, below 0.0001.

### Functional Annotation

Functional annotation of contigs was performed by searching against NCBI's non-redundant protein sequence database (NR), the Arabidopsis predicted protein sequence database and the Streptophyte predicted protein database retrieved from UniProt, using BLASTx. GO functional categories were assigned to differentially expressed genes by homology using Blast2GO (BioBam, Spain). Hierarchical clustering analysis was performed and visualized using Multiple Experiment Viewer version 4.8 34. GO-term enrichment analysis was performed and plotted using Fischer's exact test implemented in Blast2GO.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

C_TR78450 cDNA sequence
SEQ ID NO: 1
TGTCCTCACACGCAGACAAAAAACTATATATAGTTCATCCTTTAGATATT
ATTCACTTAAAACAAAACCAAAACACTTCACTCACAACTTGCAAATAATA
AACTATAACAATTCACTGAACAAACTACAACCGAAAACAAATTATGCTAC
TTATGGACAACTCTTTAGCTTCTGATTGCAGCACCGGGAAACTAATGCTC
GCTTCGCAAAACCCGAAGAAGCGAGCGGGGAGAACGAAGTTCAAAGAGAC
TCGACACCCGGTTTACCGGGGAGTGAGGATAAGAAACTCCGGAAAATGGG
TGTGTGAGGTGAGAGAACCAAATAAGAAATCGAGAGTGTGGCTGGGGACA
TACCCGACTGCCGAAATGGCGGCCCGAGCACATGATGTGGCGGTTTTGGC
AATGAGGGGACGGTCGGCTTGTTTAAATTTTGCTGACTCGGTTTGGCGGC
TGCCAGTACCCGAGTCCAGCAATGTGAAAGATATACAAAAGGCGGCTGCG
GAGGCGGCCGAGGCGTTCCGAGAGACGGAGGATATTGCGGTGGATGTGGA
GGTGGTGGTGGTGGAAGCGAATGAGGTGCCGGAAGTCGTGGTTTATGCAG
ATGAGGAGGAGATGTTTGGAATGCCGGGATTTATTGCCAGCATGGCGGAA
GGACTCATGGTGTCGCCACCTCAGATGGTGGGGTATGCTAACTTTGTGAA
TAATGTGGATTTTTGGGAAGACTTGTCTTTATGGAGTTTTTAGGGTGTTA
TATTGTTTTCTTATTTTAGAGTAAGATACATTTTGTTTGTTTAGGTAATG
GCTTAAACCTTTTTGTACACAAGCCAATATTGAAGTACATATATATCTTC
GAATAGAGATCAATCTTTTATTTAACATAGTAGTTAATAATAGTTAAGAA
TGATATGTGATTGCTT
C_TR78450 protein sequence encoded by SEQ ID NO: 1
SEQ ID NO: 2
MLLMDNSLASDCSTGKLMLASQNPKKRAGRTKFKETRHPVYRGVRIRNSG
KWVCEVREPNKKSRVWLGTYPTAEMAARAHDVAVLAIVIRGRSACLNFAD
SVWRLPVPESSNVKDIQKAAAEAAEAFRETEDIAVDVEVVVVEANEVPEV
VVYADEEENIFGMPGFIASMAEGLMVSPPQMVGYANFVNNVDFWEDLSLW
SF*
C_TR113762 cDNA sequence
SEQ ID NO: 3
AACACCTTCACAAACTCAACCATAAATAGGGCCACATAACCCATCGTTCC
TTCCCTTTCCTCCTCCCACCTTTCTTCCTATATTAAACGTTTTTCCGGCA
AAATGTTGTCTGAAGTTTTCGCCGTCGGCACCCATCTCTTCCAGGAGGAA
GCCACGTTTCTTGAAACCGGTTTCACTCCTTGGGACCCACAACAAACCCC
GGTTCTTGTTCACTCAGAACAAGAACAAGAACCGGTGTTTTCTATTTCCA
GCTCAGACAACTCCACTCCTAAGCCCAAAACCTCGGATCCACTCAACGCG
ATGGAAGAGCGTAGGCGAAGACGCAAGATATCCAACCGCGAGTCCGCAAG
GAGATCCCGGATGAGAAAACAAAAGCATTTGGAGGACATGAGGAGGCAAT
TGAACCGTCTTAAGACCGAAAACCGGGACCTAATGAACCGGTTACGGTCC
GTTAACCTCCATGGGAAACTCGTACGACACGAAAACCAGCGGCTCGTGTC
TGAATCCGTTATGTTGCAACAGAAGTTACGGAACATACGTCACGCGCTAC
ACCTCCGACAGCTTCAACACCAGTTACTCCAGTCTGCATGGCCTTGCAAT
AATAATAACGTACCCATGTATAACACCTATGAACAAAACCCACCATCATT
AATCACATAAAGAGTTAAATTATATAAATAATTGATAAATATGAACCTAA
TTACGTGCATGAAAGCAATCTCAATATCTAATAATATAGATCATCATTGT
AAATTCGAATCACGTAGGACAGGAGGGATTTTTTTTTATTTATTTTTTTT
TTTTTT
C_TR113762 protein sequence encoded by SEQ ID NO: 3
SEQ ID NO: 4
MLSEVFAVGTHLFQEEATFLETGFTPWDPQQTPVLVHSEQEQEPVFSISS
SDNSTPKPKTSDPLNAMEERRRRRKISNRESARRSRMRKQKHLEDMRRQL
NRLKTENRDLMNRLRSVNLHGKLVRHENQRLVSESVMLQQKLRNIRHALH
LRQLQHQLLQSAWPCNNNNVPMYNTYEQNPPSLIT*
C_TR132611 cDNA sequence
SEQ ID NO: 5
ATGCGTATGAGCTGCAATGGTTGTCGAGTCCTTCGTAAGGGCTGCAGTGA
AAACTGCAGCATCAGACCATGTTTGCAATGGATCAAGTCGCCTGAATCTC
AAGCTAACGCCACCGTGTTTCTCGCTAAGTTCTACGGCCGTGCTGGACTT
ATGAACCTTATTAACGCCGGCCCCGAACACCTCCGCCCTGCGATCTTCAG
GTCACTATTGTACGAGGCATGTGGTCGGATCGTGAACCCAATCTACGGAT
CAGTCGGGTTATTATGGTCGGGTAGTTGGCAGCTTTGTCAAAATGCAGTG
GAGGCTGTTCTTCAAGGACATCCGATCATTCAAATAACATCCGACACAGC
AGAAACAAACAACGGTCCACCATTCAAAGCATATGACATCCGTCACATAT
CCAAAGACGAAAACTCAGCCGGGTCAAGCGAGCTTCACCGGGTCAGAACC
CGGGGCCGGTTCAAACGGTCTGGTACGAAAGGGAAAGCGAGTCGGGTTTG
GATCGGGTCAAGGGAAGAGGAGGAGTCGGGTCTAAACGAGAACAACAATA
ACAATAATAATAGTAATAGTAATAGTAATAATAATAATAATAATGACTTG
TCGAGCCATGAGTCGGCTTTAAGCCATCAGTCTGAGGTGGCGCATGTGGT
GGAAGGTGAGAGCCGTGAAGTGGTGGAGGAAAGCTTGGAAACTTCGCCGG
CTAAAAAGCCGGCTGAAAGTGAAGCCGATGAGGTGGTGGGGAAAATAGAG
CTTGAGCTGACGTTGGGTCATGAGCTGGTTGATAAGGCTAAAAGTAAAGA
GGTTGTTGTAGCTGCGGCTTGCGAGGACGCTGATGATGGAGCCGACTTGA
ATCTGAGTCTGGATTACTCGGCTTGA
C_TR132611 protein sequence encoded by SEQ ID
NO: 5
SEQ ID NO: 6
MRIVISCNGCRVLRKGCSENCSIRPCLQWIKSPESQANATVFLAKFYGRA
GLMNLINAGPEHLRPAIFRSLLYEACGRIVNPIYGSVGLLWSGSWQLCQN
AVEAVLQGHPIIQITSDTAETNNGPPFKAYDIRHISKDENSAGSSELHRV
RTRGRFKRSGTKGKASRVWIGSREEEESGLNESNSNSNNNNNNDLSSHES
ALSHQSEVAHVVEGESREVVEESLETSPAKKPAESEADEVVGKIELELTL
GHELVDKAKSKEVVVAAACEDADDGADLNLSLDYSA

What is claimed is:

1. A method of engineering a rubber-producing plant that is a member of the family Asteraceae to increase latex production, the method comprising:
introducing an expression cassette into the rubber-producing plant, wherein the expression cassette comprises a polynucleotide encoding a rubber production transcription factor operably linked to a promoter and further, wherein, the rubber production transcription factor comprises an amino acid sequence having at least 90% identity to SEQ ID NO:2, SEQ ID NO:4, or SEQ ID NO:6;
culturing the rubber producing plant under conditions in which the transcription factor is expressed; and
selecting a plant that has increased rubber production compared to a counterpart wildtype rubber-producing plant.

2. The method of claim 1, wherein the rubber-producing plant is Russian dandelion or guayule.

3. The method of claim 1, wherein the rubber-producing plant is guayule.

4. The method of claim 1, wherein the transcription factor comprises an amino acid sequence having at least 95% identity to SEQ ID NO:2, SEQ ID NO:4, or SEQ ID NO:6.

5. The method of claim 1, wherein the transcription factor comprises the amino acid sequence of SEQ ID NO:2, SEQ ID NO:4, or SEQ ID NO:6.

6. The method of claim 1, wherein the promoter is a stem-specific promoter.

7. The method of claim 1, wherein the promoter is an inducible promoter.

8. A plant engineered by the method of claim 1, or a progeny of the plant that comprises the expression cassette.

9. A method of obtaining latex, the method comprising extracting latex from a plant engineered by the method of claim 1.

10. The method of claim 9, wherein the transcription factor comprises an amino acid sequence having at least 95% identity to SEQ ID NO:2, SEQ ID NO:4, or SEQ ID NO:6.

11. The method of claim 9, wherein the transcription factor comprises the amino acid sequence of SEQ ID NO:2, SEQ ID NO:4, or SEQ ID NO:6.

12. The method of claim 9, wherein the promoter is a stem-specific promoter.

13. The method of claim 9, wherein the promoter is an inducible promoter.

14. The method of claim 9, wherein the promoter is a native promoter of a rubber biosynthetic gene.

15. A guayule plant comprising an expression cassette that comprises a polynucleotide encoding a rubber production transcription factor operably linked to a heterologous promoter, wherein the rubber production transcription factor comprises an amino acid sequence having at least 90% identity to SEQ ID NO:2, SEQ ID NO:4, or SEQ ID NO:6.

16. The guayule plant of claim 15, wherein the transcription factor comprises an amino acid sequence having at least 95% identity to SEQ ID NO:2, SEQ ID NO:4, or SEQ ID NO:6.

17. The guayule plant of claim 15, wherein the transcription factor comprises the amino acid sequence of SEQ ID NO:2, SEQ ID NO:4, or SEQ ID NO:6.

18. The guayule plant of claim 15, wherein the heterologous promoter is a stem-specific promoter.

19. The guayule plant of claim 15, wherein the heterologous promoter is an inducible promoter.

20. The guayule plant of claim 15, wherein the heterologous promoter is a native promoter of a rubber biosynthetic gene.


SEQUENCE LISTING (6 sequences)

SEQ ID NO: 1 - DNA, 916 bp, Parthenium argentatum
tgtcctcacacgcagacaaaaaactatatatagttcatcctttagatattattcacttaaaacaaaaccaaaacacttcactcacaacttgcaaataataaactataacaattcactgaacaaactacaaccgaaaacaaattatgctacttatggacaactctttagcttctgattgcagcaccgggaaactaatgctcgcttcgcaaaacccgaagaagcgagcggggagaacgaagttcaaagagactcgacacccggtttaccggggagtgaggataagaaactccggaaaatgggtgtgtgaggtgagagaaccaaataagaaatcgagagtgtggctggggacatacccgactgccgaaatggcggcccgagcacatgatgtggcggttttggcaatgaggggacggtcggcttgtttaaattttgctgactcggtttggcggctgccagtacccgagtccagcaatgtgaaagatatacaaaaggcggctgcggaggcggccgaggcgttccgagagacggaggatattgcggtggatgtggaggtggtggtggtggaagcgaatgaggtgccggaagtcgtggtttatgcagatgaggaggagatgtttggaatgccgggatttattgccagcatggcggaaggactcatggtgtcgccacctcagatggtggggtatgctaactttgtgaataatgtggatttttgggaagacttgtctttatggagtttttagggtgttatattgttttcttattttagagtaagatacattttgtttgtttaggtaatggcttaaacctttttgtacacaagccaatattgaagtacatatatatcttcgaatagagatcaatcttttatttaacatagtagttaataatagttaagaatgatatgtgattgctt

SEQ ID NO: 2 - protein, 199 aa, Parthenium argentatum
MLLMDNSLASDCSTGKLMLASQNPKKRAGRTKFKETRHPVYRGVRIRNSGKWVCEVREPNKKSRVWLGTYPTAEMAARAHDVAVLAMRGRSACLNFADSVWRLPVPESSNVKDIQKAAAEAAEAFRETEDIAVDVEVVVVEANEVPEVVVYADEEEMFGMPGFIASMAEGLMVSPPQMVGYANFVNNVDFWEDLSLWSF

SEQ ID NO: 3 - DNA, 806 bp, Parthenium argentatum
aacaccttcacaaactcaaccataaatagggccacataacccatcgttccttccctttcctcctcccacctttcttcctatattaaacgtttttccggcaaaatgttgtctgaagttttcgccgtcggcacccatctcttccaggaggaagccacgtttcttgaaaccggtttcactccttgggacccacaacaaaccccggttcttgttcactcagaacaagaacaagaaccggtgttttctatttccagctcagacaactccactcctaagcccaaaacctcggatccactcaacgcgatggaagagcgtaggcgaagacgcaagatatccaaccgcgagtccgcaaggagatcccggatgagaaaacaaaagcatttggaggacatgaggaggcaattgaaccgtcttaagaccgaaaaccgggacctaatgaaccggttacggtccgttaacctccatgggaaactcgtacgacacgaaaaccagcggctcgtgtctgaatccgttatgttgcaacagaagttacggaacatacgtcacgcgctacacctccgacagcttcaacaccagttactccagtctgcatggccttgcaataataataacgtacccatgtataacacctatgaacaaaacccaccatcattaatcacataaagagttaaattatataaataattgataaatatgaacctaattacgtgcatgaaagcaatctcaatatctaataatatagatcatcattgtaaattcgaatcacgtaggacaggagggatttttttttatttatttttttttttttt

SEQ ID NO: 4 - protein, 185 aa, Parthenium argentatum
MLSEVFAVGTHLFQEEATFLETGFTPWDPQQTPVLVHSEQEQEPVFSISSSDNSTPKPKTSDPLNAMEERRRRRKISNRESARRSRMRKQKHLEDMRRQLNRLKTENRDLMNRLRSVNLHGKLVRHENQRLVSESVMLQQKLRNIRHALHLRQLQHQLLQSAWPCNNNNVPMYNTYEQNPPSLIT

SEQ ID NO: 5 - DNA, 876 bp, Parthenium argentatum
atgcgtatgagctgcaatggttgtcgagtccttcgtaagggctgcagtgaaaactgcagcatcagaccatgtttgcaatggatcaagtcgcctgaatctcaagctaacgccaccgtgtttctcgctaagttctacggccgtgctggacttatgaaccttattaacgccggccccgaacacctccgccctgcgatcttcaggtcactattgtacgaggcatgtggtcggatcgtgaacccaatctacggatcagtcgggttattatggtcgggtagttggcagctttgtcaaaatgcagtggaggctgttcttcaaggacatccgatcattcaaataacatccgacacagcagaaacaaacaacggtccaccattcaaagcatatgacatccgtcacatatccaaagacgaaaactcagccgggtcaagcgagcttcaccgggtcagaacccggggccggttcaaacggtctggtacgaaagggaaagcgagtcgggtttggatcgggtcaagggaagaggaggagtcgggtctaaacgagaacaacaataacaataataatagtaatagtaatagtaataataataataataatgacttgtcgagccatgagtcggctttaagccatcagtctgaggtggcgcatgtggtggaaggtgagagccgtgaagtggtggaggaaagcttggaaacttcgccggctaaaaagccggctgaaagtgaagccgatgaggtggtggggaaaatagagcttgagctgacgttgggtcatgagctggttgataaggctaaaagtaaagaggttgttgtagctgcggcttgcgaggacgctgatgatggagccgacttgaatctgagtctggattactcggcttga

SEQ ID NO: 6 - protein, 291 aa, Parthenium argentatum
MRMSCNGCRVLRKGCSENCSIRPCLQWIKSPESQANATVFLAKFYGRAGLMNLINAGPEHLRPAIFRSLLYEACGRIVNPIYGSVGLLWSGSWQLCQNAVEAVLQGHPIIQITSDTAETNNGPPFKAYDIRHISKDENSAGSSELHRVRTRGRFKRSGTKGKASRVWIGSREEEESGLNENNNNNNNSNSNSNNNNNNDLSSHESALSHQSEVAHVVEGESREVVEESLETSPAKKPAESEADEVVGKIELELTLGHELVDKAKSKEVVVAAACEDADDGADLNLSLDYSA